(19) United States
(12) Patent Application Publication
(10) Pub. No.: US 2007/0178074 A1
(43) Pub. Date: Aug. 2, 2007

(54) Chondrocyte Culture Formulations

(76) Inventors: Dan-Ning Hu, Bayside, NY (US); Steven McCormick, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/539,464

(22) Filed: Oct. 6, 2006

Related U.S. Application Data
(63) Continuation of application No. PCT/US05/13515, filed on Apr. 19, 2005.
(60) Provisional application No. 60/564,672, filed on Apr. 21, 2004.

(51) Int. Cl. A61K 35/12; C12N 5/08; C12N 5/06

(52) U.S. Cl. 424/93.7; 435/325; 435/366

(57) ABSTRACT

The present invention provides methods and compositions for providing graft recipients with chondrocytes. Specifically, the methods and compositions of the invention provide for populations of chondrocytes that may be isolated from a patient and cultured in vitro to generate a proliferating population of chondrocytes that exhibit great proliferation capacity. The invention is based, in part, on the discovery of a culture medium having serum, insulin and various growth factors for culturing chondrocytes that exhibit increased proliferative capacity. The invention provides novel in vitro methods for culturing chondrocytes, including those isolated from the cartilage of a healthy subject, to generate a proliferating population of chondrocytes. The methods and compositions of the invention may be used for transplantation to treat patients having damaged cartilage.

### CROSS-REFERENCE TO RELATED APPLICATIONS

The present application claims the benefit of priority to U.S. Provisional Application No. 60/564,672, filed Apr. 21, 2004, the contents of which are incorporated herein in their entirety by reference.

### FIELD OF THE INVENTION

The present invention relates to cell culture compositions such as compositions for culturing chondrocytes. The present invention also relates to methods for culturing chondrocytes and cell cultures comprising chondrocytes.

### BACKGROUND OF THE INVENTION

The articulating surface of the knee is covered with articular cartilage. Normal articular cartilage (hyaline cartilage) is composed of type II collagen fibers and mucopolysaccharides, both synthesized by chondrocytes. Functional articular cartilage is critical to proper joint function. Defects in the articular cartilage can result in pain, locking, and other activity-limiting symptoms. Unfortunately, articular cartilage heals poorly. The repair tissue often is fibrocartilaginous and, with time, tends to deteriorate into fibrous tissue with poor mechanical properties. Past efforts to promote re-growth of articular cartilage include simple surgical technical techniques such as curettage and more complex grafting techniques to replace the articular surface (Minas T, Nehrer S. Current concepts in the treatment of articular cartilage defects. Orthopedics 1997;20:525-38).

Autologous chondrocyte transplantation provides another option for the treatment of articular cartilage damage and was introduced by Brittberg (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95). The treatment promotes the restoration of loadable hyaline articular cartilage by transplantation of autologous chondrocytes. Chondrocytes were harvested arthroscopically from an uninjured minor load-bearing area of the damaged knee, cultured for several weeks to increase the cell number by 10-fold to 50-fold, and transplanted to the area of articular cartilage damage under a sutured periosteal flap. Since then, many patients suffering from joint injury and diseases (mainly the knee) have been treated using autologous chondrocyte transplantation with positive results (Gillogly S D, Voight M, Blackburn T. Treatment of articular cartilage defects of the knee with autologous chondrocyte implantation. J Orthop Sports Phys Ther 1998;28:241-51; Peterson L, Minas T, Brittberg M, Nilsson A, Sjogren-Jansson E, Lindahl A. Two- to 9-year outcome after autologous chondrocyte transplantation of the knee. Clin Orthop 2000:212-34; Peterson L, Brittberg M, Kiviranta I, Akerlund E L, Lindahl A. Autologous chondrocyte transplantation. Biomechanics and long-term durability. Am J Sports Med 2002;30:2-12).

One of the most important steps for the success of autologous chondrocyte transplantation is the culture of chondrocytes. Several types of chondrocyte culture media for the culture of chondrocytes that provide autologous transplantation of chondrocytes have been described as discussed below.

The earliest culture medium used for the culture of chondrocytes for transplantation was reported by Brittberg (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95), which consists Ham's F12 medium (F12 medium), supplemented with 15% autologous serum, HEPES buffer (10 mM), gentamicin (50 μg/ml), amphotericin B (2 μg/ml) and L-ascorbic acid (50 μg/ml). This formulation was modified later by Peterson (Peterson L, Minas T, Brittberg M, Nilsson A, Sjogren-Jansson E, Lindahl A. Two- to 9-year outcome after autologous chondrocyte transplantation of the knee. Clin Orthop 2000:212-34) who used F12/Dulbecco's Modified Eagle Medium (DMEM), changed the concentration of autologous serum to 10%, omitted the HEPES buffer, and added the glutamine (Peterson L, Brittberg M, Kiviranta I, Akerlund E L, Lindahl A. Autologous chondrocyte transplantation. Biomechanics and long-term durability. Am J Sports Med 2002;30:2-12). This modified medium has been used for transplantation of chondrocytes in Sweden. However, no quantitative studies have been reported on the growth aspects of chondrocytes cultured with this medium. Therefore, the effects of this medium on the growth of chondrocytes are difficult to evaluate.

U.S. Pat. No. 6,150,163 discloses the use of a culture medium for culturing human chondrocytes (Genzyme Corporation). This culture medium comprises DMEM, F12 and RPMI 1640 medium (1:1:1), with 1% ITS, penicillin (100 U/ml), streptomycin (100 μg/ml), hydrocortisone (40 μg/ml), bFGF (10 μg/ml), IGF-1 (1 ng/ml) and fibronectin 5 μg/ml. Notably, this culture medium appears to be serum-free.

It has been reported that basic fibroblast growth factor (bFGF), a growth factor, can stimulate the proliferation of chondrocytes in vitro (Martin I, Vunjak-Novakovic G, Yang J, Langer R, Freed L E. Mammalian chondrocytes expanded in the presence of fibroblast growth factor 2 maintain the ability to differentiate and regenerate three-dimensional cartilaginous tissue. Exp Cell Res 1999;253:681-8). However, the reports on the proliferation enhancing effect of bFGF on chondrocytes have been conflicting and fail to report any successful chondrocyte transplantation using chondrocytes cultured with media containing bFGF (Trippel S B. Growth factor actions on articular cartilage. J Rheumatol Suppl 1995;43:129-32; de Haart M, Marijnissen W J, van Osch G J, Verhaar J A. Optimization of chondrocyte expansion in culture. Effect of TGF beta-2, bFGF and L-ascorbic acid on bovine articular chondrocytes. Acta Orthop Scand 1999;70:55-61).

Platelet-derived growth factor (PDGF) has been reported to have a growth stimulation effects on the chondrocytes in vitro (Toolan B C, Frenkel S R, Pachence J M, Yalowitz L, Alexander H. Effects of growth-factor-enhanced culture on a chondrocyte-collagen implant for cartilage repair. J Biomed Mater Res 1996;31:273-80; Guerne P A, Blanco F, Kaelin A, Desgeorges A, Lotz M. Growth factor responsiveness of human articular chondrocytes in aging and development. Arthritis Rheum 1995;38:960-8). These studies fail to report any successful chondrocyte transplantation using chondrocytes cultured with media containing PDGF.

Hepatocyte growth factor (HGF) is a multi-function growth factor. It has been reported that the HGF receptor, c-met, is expressed in articular chondrocytes (Bau B, McKenna L A, Soeder S, Fan Z, Pecht A, Aigner T. Hepatocyte growth factor/scatter factor is not a potent regulator of anabolic and catabolic gene expression in adult human articular chondrocytes. Biochem Biophys Res Commun 2004;316:984-90). Little is known of the effect of HGF on proliferation of chondrocytes in vitro. One report indicated that HGF can stimulate DNA synthesis in rabbit chondrocytes (Takebayashi T, Iwamoto M, Jikko A, et al. Hepatocyte growth factor/scatter factor modulates cell motility, proliferation, and proteoglycan synthesis of chondrocytes. J Cell Biol 1995;129:1411-9). This study did not report any culture media containing HGF for growing chondrocytes.

### SUMMARY OF THE INVENTION

The present invention relates to methods and compositions for providing transplantation recipients with chondrocytes. In particular, the present invention provides compositions and methods for culturing chondrocytes using the compositions. For example, chondrocytes may be isolated from a patient, cultured in a specified medium in vitro to generate a proliferating population of chondrocytes, and re-introduced into the patient. The invention is based, in part, on the discovery of culture media for culturing chondrocytes, wherein chondrocytes cultured in such media exhibit increased proliferative capacity. The invention provides novel in vitro methods for culturing chondrocytes, including those isolated from the cartilage of a patient, to generate a proliferating population of chondrocytes. The methods and compositions of the invention may be used for transplantation for patients in need of chondrocyte transplantation including, without limitation, patients having defects of cartilage.

The compositions of the invention relate to a culture medium comprising a basal medium that is supplemented with sera (e.g., bovine serum), hormones (e.g., insulin), and growth factors (e.g., HGF and PDGF). In particular, the culture medium comprises basal media, sera, antibiotics, hormones, and growth factors. In an embodiment, the culture medium comprises both PDGF and HGF.

Any serum known in the art may be used in accordance with the present invention, including but not limited to human serum, bovine serum, newborn bovine serum, fetal bovine serum, porcine serum, equine serum, and combinations thereof.

Any basal medium known in the art may be used in accordance with the present invention, including but not limited to Ham's F12, RPMI, DMEM, and combinations thereof, and in any ratio thereof. In a specific embodiment, the basal medium comprises Ham's F12 and RPMI in a ratio of about 1:1.

Any growth factor known in the art may be used in accordance with the present invention, including but not limited to bFGF, HGF, PDGF, EGF, IGF, TGF-β, and combinations thereof.

Any hormone known in the art may be used in accordance with the present invention, including but not limited to androgen, mineralocorticoid, glucocortoid, insulin, hydrocortisone, estradiol, progestin, growth hormone, and combinations thereof.

The present invention also relates to methods of culturing chondrocytes for transplantation. For example, a proliferating population of chondrocytes can be obtained by isolating chondrocytes from a donor (e.g., from a cartilage sample in vivo), and culturing the isolated chondrocytes using a culture medium of present invention. The chondrocytes can be harvested from a cartilage sample by enzymatically treating the cartilage to dissociate chondrocytes from the sample.

The present invention also provides methods of providing a subject in need of cartilage repair with a proliferating population of chondrocytes. The method includes isolating autologous chondrocytes from the subject, culturing the isolated autologous chondrocytes in a culture medium of present invention to obtain a proliferating population of cultured chondrocytes, and introducing the cultured chondrocytes to the subject. The cultured chondrocytes may be applied to surface of the cartilage requiring repair, or they may be introduced into the local environment of the cartilage.

In another embodiment, the invention provides transplantation methods of treating cartilage defects utilizing cultured autologous or heterologous chondrocytes by isolating chondrocytes from an individual, culturing the isolated chondrocytes in a culture medium of present invention to obtain a population of chondrocytes with enhanced proliferative capabilities, and transplanting the cultured chondrocytes onto the surface, or in the local environment, of the defective cartilage.

In a specific embodiment of the invention, cultured chondrocytes are genetically engineered, prior to transplantation, to enable them to express one or more growth factors, cytokines, extracellular matrix proteins, or other biologically active molecules. In this way, any new tissue derived from the transplanted chondrocytes will express or overexpress the desired biologically active molecule.

In another specific embodiment of the invention, chondrocytes may be cultured with a matrix before the transplantation to improve cell adhesion, proliferation and/or differentiation. For example, cell differentiation may be triggered or continued upon transplantation. Chondrocytes cultured with exogenous matrix or matrix produced by the same cells can also be used for transplantation.

The present invention also provides compositions that include chondrocytes cultured in a medium comprising basal media, sera, antibiotics, hormones, and growth factors. Such compositions preferably include PDGF and HGF. The chondrocytes of these compositions exhibit greatly increased proliferative capacity compared to other known chondrocyte cultures. For example, the cultured chondrocytes exhibit many more divisions before senescence than do other chondrocyte cultures. The cultured chondrocytes of the invention divide at least 10-40 times before senescence, e.g., the cultured chondrocytes may divide 10, 12, 14, 16, 18, 20, 22, 24, 26, 28, 30, 32, 34, 36, 38, or 40 times before senescence.

### BRIEF DESCRIPTIONS OF THE DRAWINGS

FIG. 1. Effects of various basic culture media on the growth of cultured articular chondrocytes. Cells were cultured with F12 medium (F12) (control); Dulbecco's Modified Eagle Medium (DMEM); RPMI 1640 (RPMI); F12 and DMEM (1:1) (FD); F12 and RPMI (1:1) (FR); F12, DMEM and RPMI (1:1:1) (FDR) (all supplemented with 10% FBS) and cultured for 6 days. Cell number was counted and compared with the controls. The results are expressed as the percentages of the controls (3 wells in each group, Mean±SD).

FIG. 2. Effects of serum on the growth of cultured articular chondrocytes. Cells were cultured with F12 medium or supplemented with various concentrations of FBS and cultured for 6 days. Cell number was counted and compared with the controls (without serum). The results are expressed as the percentages of the controls (3 wells in each group, Mean±SD).

FIG. 3. Effects of bFGF on the growth of cultured articular chondrocytes. Cells were cultured with FR culture medium with 10% FBS (control) or supplemented with various concentrations of bFGF (ng/ml) and cultured for 6 days. Cell number was counted and compared with the controls. The results are expressed as the percentages of the controls (3 wells in each group, Mean±SD).

FIG. 4. Effects of HGF on the growth of cultured articular chondrocytes. Cells were cultured with FR culture medium with 10% FBS (control) or supplemented with various concentrations of HGF (ng/ml) and cultured for 6 days. Cell number was counted and compared with the controls. The results are expressed as the percentages of the controls (3 wells in each group, Mean±SD).

FIG. 5. Effects of PDGF-AB on the growth of cultured articular chondrocytes. Cells were cultured with FR culture medium with 10% FBS (control) or supplemented with various concentrations of PDGF (ng/ml) and cultured for 6 days. Cell number was counted and compared with the controls. The results are expressed as the percentages of the controls (3 wells in each group, Mean±SD).

FIG. 6. Effects of insulin on the growth of cultured articular chondrocytes. Cells were cultured with FR culture medium with 10% FBS (control) or supplemented with various concentrations of insulin (μg/ml) and cultured for 6 days. Cell number was counted and compared with the controls. The results are expressed as the percentages of the controls (3 wells in each group, Mean±SD).

FIG. 7. Effects of combination of various supplements on the growth of cultured articular chondrocytes. Cells were cultured with FR culture medium with 10% FBS (control) or supplemented with bFGF 15 μg/ml (F), bFGF 15 μg/ml with HGF 100 μg/ml (FH), bFGF 15 μg/ml with HGF 100 μg/ml and PDGF 50 μg/ml (FHP), or FHP with insulin 20 ug/ml (FHPI), and cultured for 6 days. Cell number was counted and compared with the controls. The results are expressed as the percentages of the controls (3 wells in each group, Mean±SD).

FIG. 8. Comparison of articular chondrocytes cultured with various culture media. Cells were cultured with medium described in U.S. Pat. No. 6,150,163 (G), F12 medium with 10% serum (10% F12) and Hu60 medium (Hu60) for 6 days. Cell number was counted and compared. The results are expressed as the percentages of the control (10% F12 group) (3 wells in each group Mean±SD).

FIG. 9. Comparison of various media on their long-term effects on cell growth of cultured articular chondrocytes. In 3 cell lines of chondrocytes, cells were seeded into 3 flasks at the second subculture and cultured with medium described in U.S. Pat. No. 6,150,163 (G), F12 medium with 10% serum (10% F12) or Hu60 medium (Hu60) separately. Cells were incubated and subculture until senescence. Population doubling and period of culture before senescence were expressed as the percentages of the 10% F12 group (3 cell lines in each group, Mean±SD).

### DETAILED DESCRIPTION OF THE INVENTION

The present invention provides novel compositions and methods for culturing chondrocytes and the use of such chondrocytes for transplantation. For example, the methods and compositions of the invention may be used to treat cartilage damage.

The compositions of the invention relate to a culture medium comprising basal medium that is supplemented with sera, growth factors, and hormones. The present invention is based, in part, on the observation that chondrocytes isolated and cultured in media of the present invention have greatly enhanced proliferative capacity. The chondrocytes, include but are not limited to articular chondrocytes, auricular chondrocytes, nasal chondrocytes, chondrocostal chondrocytes, or mixtures thereof.

In a specific embodiment, the culture medium of the present invention comprises the components listed below.

Formula of Hu60 culture medium:
Ham's F-12 nutrient mixture (F-12 medium) with RPMI-1640 medium (RPMI medium) 1:1
Fetal bovine serum (FBS) 10%
Basic fibroblast growth factor (bFGF) 15 μg/ml
Hepatocyte growth factor (HGF) 100 μg/ml
Platelet-released growth factor-AB (PDGF-AB) 50 μg/ml
Insulin 20 μg/ml
Glutamine 2 mM
Gentamicin 50 μg/ml.

F-12 medium, RPMI medium, FBS and gentamicin were obtained from the GIBCO™ (Carlsbad, Calif.).

bFGF, HGF and PDGF-AB were obtained from the PeproTech™ (Rocky Hill, N.J.). Insulin was obtained from the SIGMA™ (St. Louis, Mo.).

Glutamine is added to F-12 medium and RPMI-1640 medium at 1:1 proportion. Gentamicin is added to the culture medium to obtain a final concentration of 50 μg/ml. Fetal bovine serum is added to the medium to obtain a 10% concentration (volume/volume). The medium containing FBS and gentamicin is stored at 4° C. and prepared fresh every two weeks. bFGF is dissolved in F-12 medium to 1,500 μg/ml and stored in small vials at −70° C. The stored bFGF solution is added to the culture medium to obtain a final concentration of 15 μg/ml once a week. HGF is dissolved in F-12 medium to 10,000 μg/ml and stored in small vials at −70° C. The stored HGF solution is added to the culture medium to obtain a final concentration of 100 μg/ml once a week. PDGF-AB is dissolved in F-12 medium to 5,000 μg/ml and stored in small vials at −70° C. The stored PDGF-AB solution is added to the culture medium to obtain a final concentration of 50 μg/ml once a week. Insulin is dissolved in F-12 medium to 2 mg/ml and stored in small vials at −70° C. The stored insulin solution is added to the culture medium to obtain a final concentration of 20 μg/ml once a week.

Isolation and Culturing of Chondrocytes

Chondrocytes may be obtained from a variety of different donor sources including autologous, allogenic, or heterologous sources. In an embodiment, autologous chondrocytes are obtained from the subject who is to receive the chondrocytes (autologous graft). This approach is especially advantageous since the immunological rejection of foreign tissue and/or a graft versus host response is avoided. In another embodiment of the invention, allogenic chondrocytes may be obtained from donors who are genetically related to the recipient and share the same transplantation antigens on the surface of their chondrocytes. Alternatively, if a related donor is unavailable, chondrocytes from antigenetically matched donors may be used. Low-temperature or other methods may be used to reduce the antigenicity of chondrocytes so even allogenic chondrocyte transplantation is possible (Chen F S, Frenkel S R, Di Cesare P E. Chondrocyte transplantation and experimental treatment options for articular cartilage defects. Am J Orthop 1997;26:396S-406; Schreiber R E, Iten-Kirby B M, Dunkelman N S, et al. Repair of osteochondral defects with allogenic tissue engineered cartilage implants. Clin Orthop 1999;367S:382-95).

Chondrocytes may be obtained from the cartilage using a variety of different methods. In the case of articular chondrocytes, the cartilage specimen can be obtained through an arthroscope from a minor load-bearing area on the upper medial femoral condyle of the damaged knee. A small piece of full thickness cartilage (5 mm wide by 10 mm in length, 200-500 mg) can be cut by a ring curette or sharp gouges. The cartilage specimen is placed in a sterile vial containing 0.9% NaCl or other solutions (Hank's solution, F12 medium etc) with same osmolarity and has a pH ranged from 6.5-7.5. The cartilage is transferred to the cell culture laboratory immediately (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Minas T, Brittberg M, Nilsson A, Sjogren-Jansson E, Lindahl A. Two- to 9-year outcome after autologous chondrocyte transplantation of the knee. Clin Orthop 2000:212-34; Peterson L, Brittberg M, Kiviranta I, Akerlund E L, Lindahl A. Autologous chondrocyte transplantation. Biomechanics and long-term durability. Am J Sports Med 2002;30:2-12; Minas T, Peterson L. Advanced techniques in autologous chondrocyte transplantation. Clin Sports Med 1999;18:13-44)

The cartilage can be washed with Hanks solution (or other buffered solution, e.g., F12 medium, etc.) supplemented with gentamicin sulfate (50 μg/ml), amphotericin (2 μg/ml) and L-ascorbic acid (50 μg/ml); minced into small pieces (1-2 mm³) and washed again with Hanks solution (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Brittberg M, Kiviranta I, Akerlund E L, Lindahl A. Autologous chondrocyte transplantation. Biomechanics and long-term durability. Am J Sports Med 2002;30:2-12).

The minced cartilage can be digested by a variety enzymes. Such enzymes include, but are not limited to, trypsin, chymotrypsin, collagenase, deoxyribonuclease, elastase and/or hyaluronidase. For example, the minced cartilage can be digested overnight (16-20 hours) in a culture flask containing F12 medium supplemented with collagenase 1 mg/ml (1200 IU/mg, SIGMA), deoxyribonuclease I (0.1 mg/ml, SIGMA) and other supplements described above. Isolated cells are collected and equal volume of 0.02% of EDTA solution can be added to stop the action of collagenase. Cell suspension is centrifuged at 1800 rpm for 6 minutes. The supernatant is discarded and the cells are resuspended with Hu60 medium, counted with a hemocytometer and seeded into 25 cm² culture flask. The fragments remaining after collagenase digestion can be incubated with collagenase solution for 2 hours. Released cells are collected, centrifuged, resuspended and seeded as described previously (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Brittberg M, Kiviranta I, Akerlund E L, Lindahl A. Autologous chondrocyte transplantation. Biomechanics and long-term durability. Am J Sports Med 2002;30:2-12; Hu D N, Yang P Y, Ku M C, et al. Isolation and cultivation of human articular chondrocytes. Kaosiung J Med Sci 2002;18:113-120).

Cells can be cultured in a CO₂-regulated incubator in a humidified 95% air/5% CO₂ atmosphere. The culture medium used is Hu60 medium. The cultures are observed daily by inverted phase-contrast microscopy. The culture medium is replaced 3 times a week. After primary cultures became confluent, the cells are detached by trypsin (0.05%)-EDTA (0.02%) solution, counted, centrifuged, re-suspended, diluted with a ratio 1:2-1:4, and seeded into culture flasks for subculture. Cells are passed routinely by a dilution of 1:2 to 1:4 at an interval for 4-7 days until adequate number of cells can be collected for transplantation (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Brittberg M, Kiviranta I, Akerlund E L, Lindahl A. Autologous chondrocyte transplantation. Biomechanics and long-term durability. Am J Sports Med 2002;30:2-12; Hu D N, Yang P Y, Ku M C, et al. Isolation and cultivation of human articular chondrocytes. Kaosiung J Med Sci 2002;18:113-120).

The medium of the invention comprises basal medium supplemented with bovine serum, various growth factors and insulin. The basal medium may be any of the standard culture medium that provides the minimal requirements to sustain the growth of cells in culture. Such basal media, include but are not limited to basal amino acid/ salt mixtures such as Ham's F12, RPMI, or DMEM. Serum is added to the media in concentrations of between 5-30%. Any type of animal serum may be used, including but not limited to, fetal calf, calf, equine, goat or human serum. Additional additives to the medium may include, for example, glucose, glutamine, vitamins and any additional additives known to those of skill in the art.

Growth factors and cytokines to be added to the basal medium include bFGF, PDGF and HGF. The addition of these growth factors to the medium was found to enhance the proliferation of chondrocytes cultured in the medium, thereby shortening the patient's wait to transplantation. Further, in patients wherein chondrocytes do not grow well in regular culture medium (with serum but without growth factors) so that the in vitro expansion of cell number required for transplantation cannot be met, adding of various growth factors and insulin to improve the growth of cells thereby facilitating transplantation.

In an embodiment of the invention, the culture medium comprises the growth factor, bFGF. The concentration of bFGF may be between 1 and 1000 μg/ml. In an embodiment of the invention, the concentration of bFGF in the media is between 10 and 100 μg/ml. In a preferred embodiment of the invention, the concentration of bFGF in the media is between 5 and 25 μg/ml. In a specific embodiment of the invention, the culture medium contains bFGF at a concentration of 15 μg/ml.

In an embodiment of the invention, the culture medium comprises the growth factor, HGF. The concentration of HGF may be between 10 to 1000 μg/ml. In an embodiment of the invention, the concentration of HGF is between 50 to 750 μg/ml. In a preferred embodiment of the invention, the concentration of HGF in the media is between 75-500 μg/ml. In a specific embodiment of the invention, the culture medium contains HGF at a concentration of 100 μg/ml.

In an embodiment of the invention, the culture medium comprises the growth factor, PDGF-AB. The concentration of PDGF may be between 10 to 1000 μg/ml. In an embodiment of the invention, the concentration of PDGF is between 20 to 200 μg/ml. In a preferred embodiment of the invention, the concentration of PDGF is between 30-100 μg/ml. In a specific embodiment of the invention, the culture medium contains PDGF at a concentration of 50 μg/ml.

The culture medium may comprise the hormone, insulin, present at a concentration between 1 and 1000 μg/ml. In an embodiment of the invention, the concentration of insulin is between 5 and 200 μg/ml. In a preferred embodiment of the invention, the concentration of insulin is between 10 and 50 μg/ml. In a specific embodiment of the invention, the culture medium contains insulin at a concentration of 20 μg/ml.

The culture medium may comprise both growth factors and hormones. Growth factors may include, but are not limited to, bFGF, HGF, PDGF, EGF, IGF and/or TGF-β, including any combination thereof. Hormones include, but are not limited to, insulin, hydrocortisone, estradiol, and/or progesterone, including any combination thereof.

Those of skill in the art will also recognize that one or more commercially available substances may be used as additives or substitutions to the medium to support the growth of chondrocytes. Such growth may be monitored using a number of different methods. For example, proliferation of cells can be monitored by cell counts using a hemocytometer or flow cytometer.

Growth of Chondrocytes

The differential capacity of cultured chondrocytes is not strongly correlated to the successful of the transplantation of chondrocytes. Chondrocytes cultured with bFGF show significant proliferation ability and de-differentiate, preserve their chondrogenic potential, after seeded to a 3D polymer scaffold, produce cartilage that is comparable to that obtained using primary chondrocytes, and differentiate better than chondrocytes not cultured with bFGF (Martin I, Vunjak-Novakovic G, Yang J, Langer R, Freed L E. Mammalian chondrocytes expanded in the presence of fibroblast growth factor 2 maintain the ability to differentiate and regenerate three-dimensional cartilaginous tissue. Exp Cell Res 1999;253:681-8). Therefore, a more important property of a culture medium for culturing chondrocytes for the eventual transplantation is the ability to enhance the proliferative ability of chondrocytes in vitro, and not necessarily to promote differentiation of the chondrocytes in vitro.

The chondrocytes cultured in the claimed culture medium exhibit greatly increased proliferative capacity compared to other known chondrocyte cultures. The cultured chondrocytes exhibit many more divisions before senescence than do other chondrocyte cultures. The cultured chondrocytes of the invention divide at least 10-40 times before senescence, e.g., 10 12, 14, 16, 18, 20, 22, 24, 26, 28, 30, 32, 34, 36, 38, or 40 times. The results in the Examples show that cultured chondrocytes are capable of dividing over 25 times before senescence.

Administration of Chondrocytes

Cultured chondrocytes may be grafted directly onto the surface of the cartilage of the subject, grafted directly onto the surface of a supporting matrix, or introduced into the local environment of the cartilage or the supporting matrix. The cultured chondrocytes may then proliferate, differentiate, and/or produce cartilage, such as hyaline cartilage.

Transplantation of chondrocytes may be performed when the culture of chondrocytes obtains a sufficient proliferating number of cells. This may occur 14-21 days after obtaining the biopsy specimen. Three days before transplantation, cell culture is subjected to sterility testing (bacterial and fungal growth). Cultured chondrocytes are detached from the flask by typsin (0.05%)-EDTA (0.02%) solution, one drop of the cell suspension is mixed with one drop of 0.4% trypan blue solution and examined under the light microscope to determine the viability of the cell. Dead cells are stained by the trypan blue, whereas the living cells exclude trypan blue. Cells are released for transplantation if cell viability is greater than 85% (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Minas T, Brittberg M, Nilsson A, Sjogren-Jansson E, Lindahl A. Two- to 9-year outcome after autologous chondrocyte transplantation of the knee. Clin Orthop 2000:212-34; Peterson L, Brittberg M, Kiviranta I, Akerlund E L, Lindahl A. Autologous chondrocyte transplantation. Biomechanics and long-term durability. Am J Sports Med 2002;30:2-12).

Cells for transplantation are centrifuged as described above and resuspended with F12 medium without supplements. The cell suspension is aspirated into a 1 ml tuberculin syringe with a 1.2 mm needle. The final volume of the cell suspension is 0.2-0.4 ml, with a total of 2×10⁶-12×10⁶ cells ((Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Minas T, Brittberg M, Nilsson A, Sjogren-Jansson E, Lindahl A. Two- to 9-year outcome after autologous chondrocyte transplantation of the knee. Clin Orthop 2000:212-34).

In an embodiment of the invention, the chondrocytes cultured in a media of the invention are administered to a patient in need of proliferating chondrocytes. The patient may require chondrocytes to repair damaged cartilage tissue, such as in the knee or other joints. The cultured chondrocytes may also be used for cosmetic purposes, such as rhinoplasty surgery.

The procedure of administration of cultured chondrocytes can be an open-joint surgery or through the arthroscope. The following described procedure is open-joint surgery. The patient is under general or spinal anesthesia, a parapatellar arthrotomy is performed. The cartilage lesion is debrided to the best cartilage available. Care is taken not to penetrate the subchondral bone plate. A periosteal flap is harvested from the proximal medial tibia. The flap is sutured to the surrounding rim of the normal cartilage with interrupted 5-0 Dexon sutures. The periosteal rim is sealed with a fibrin glue (Tisseel, Immuno AG, Austria) with the exception of one corner of the rim where the transplanted chondrocytes are injected into the defect. The joint capsule, retinnaculum and skin are sutured in separate layers and the knee is covered with an elastic bandage. Continuous passive motion is administrated for 48 hours after surgery. Rehabilitation on crutches begins with gradual weightbearing for 8 weeks, progressing to full weightbearing by 10-12 weeks (Brittberg M, Lindahl A, Nilsson A, Ohlsson C, Isaksson O, Peterson L. Treatment of deep cartilage defects in the knee with autologous chondrocyte transplantation. N Engl J Med 1994;331:889-95; Peterson L, Minas T, Brittberg M, Nilsson A, Sjogren-Jansson E, Lindahl A. Two- to 9-year outcome after autologous chondrocyte transplantation of the knee. Clin Orthop 2000:212-34; Peterson L, Brittberg M, Kiviranta I, Akerlund E L, Lindahl A. Autologous chondrocyte transplantation. Biomechanics and long-term durability. Am J Sports Med 2002;30:2-12).

The present methods and compositions may additionally employ cultured chondrocytes genetically engineered to enable them to produce a wide range of functionally active biologically active proteins including, but not limited to, growth factors, cytokines, hormones, inhibitors of cytokines, peptide growth and differentiation factors, and extracellular matrix proteins. Methods which are well known to those skilled in the art can be used to construct expression vectors containing a nucleic acid encoding the protein of interest linked to appropriate transcriptional/translational control signals (Sambrook, et al. Molecular Cloning, A Laboratory Manual, Cold Spring Harbor Laboratory, New York. 1992; Ausebel et al. Current Protocols in Molecular Biology, Greene Publishing Associates & Wiley Interscience, New York, 1989). The expression vectors may be introduced into the chondrocyte by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment (Johnston et al., Science 240:1538 (1988)).

In addition, cultured chondrocytes may be attached in vitro to a natural or synthetic matrix that provides support for the transplanted chondrocytes prior to, during, and/or post-transplantation (Minas T, Nehrer S. Current concepts in the treatment of articular cartilage defects. Orthopedics 1997;20:525-38; Jackson D W, Simon T M. Chondrocyte transplantation. Anthroscopy 1996;12:732-38). The matrix may have all the features commonly associated with being biocompatible, in that it is in a form that does not produce an adverse, or allergic reaction when administered to the recipient host. Such matrices may be formed from both natural and synthetic materials. For example the support matrix can be made from collagen, such as Type I or Type II collagen. The matrix may also be designed to allow for sustained release of growth factors over prolonged periods of time. Thus, appropriate matrices may both provide growth factors and also act as an in situ scaffolding in which the chondrocytes may proliferate and differentiate. In particular embodiments, the matrix comprises hyaluronic acid, collagen, and any combination thereof. In preferred embodiments, it is contemplated that a biodegradable matrix that is capable of being reabsorbed into the body will likely be most useful.

To improve chondrocytes adhesion to the matrix, survival, function and/or migration of the chondrocytes, the matrix may optionally be coated on its external surface with factors known in the art to promote cell adhesion, growth, survival or migration. Such factors may include cell adhesion molecules, extracellular matrix molecules or growth factors.

### EXAMPLES

These examples further describe and demonstrate embodiments within the scope of the present invention. The examples are given solely for the purpose of illustration and are not to be construed as limitations of the present invention, as many variations thereof are possible without departing from the spirit and scope of the invention.

### Culture of Articular Chondrocytes

Glutamine was added to Ham's F12 medium and RPMI 1640 medium at 1:1 proportion. Gentamicin was added to the culture medium to obtain a final concentration of 50 μg/ml. Fetal bovine serum (FBS) was added to the medium to obtain a 10% concentration (volume/volume). The medium containing FBS and gentamicin was stored at 4° C. and prepared fresh every two weeks. bFGF was dissolved in F12 medium to 1,500 μg/ml and stored in small vials at −70° C. The stored bFGF solution was added to the culture medium to obtain a final concentration of 15 μg/ml once a week. HGF was dissolved in F12 medium to 10,000 μg/ml and stored in small vials at −70° C. The stored HGF solution was added to the culture medium to obtain a final concentration of 100 μg/ml once a week. PDGF was dissolved in F12 medium to 5,000 μg/ml and stored in small vials at −70° C. The stored PDGF solution was added to the culture medium to obtain a final concentration of 50 μg/ml once a week. Insulin was dissolved in F12 medium to 2 mg/ml and stored in small vials at −70° C. The stored insulin solution was added to the culture medium to obtain a final concentration of 20 μg/ml once a week.

### Isolation and Culture of Articular Chondrocytes

Articular cartilage was collected from donors and were placed in a sterile vial containing Ca⁺⁺- and Mg⁺⁺-free Hanks solution supplemented with gentamicin (50 μg/ml) and amphotericin (2.5 μg/ml) (d-Hanks solution) and was immediately transferred to the cell culture laboratory. The cartilage was washed with d-Hanks solution, minced into small pieces (1-2 mm³) and washed again with d-Hanks solution.

The minced cartilage was immersed in 0.25% trypsin solution and incubated for 30 minutes at 37 C. Trypsin activity was stopped by adding of equal volume of F12 medium with 10% serum. The mixed solution with few released cells was withdrawn and discarded. The remaining cartilage was incubated at 37° C. with collagenase (2.0 mg/ml) in F12 medium with 10% serum for 4 hours. The isolated cells and collagenase solution were collected and equal volume of 0.02% of EDTA solution was added to stop the action of collagenase. Cell suspension was centrifuged at 1800 rpm for 6 minutes. The supernatant was discarded and the cells were re-suspended with culture medium, counted with a hemocytometer and seeded into 6-cm Falcon culture dishes. The fragments remaining after collagenase digestion were incubated with collagenase solution for 2 hours. Released cells were collected, centrifuged, re-suspended and seeded as described previously.

Cells were incubated in a CO₂-regulated incubator in a humidified 95% air/5% CO₂ atmosphere. The culture medium used was F12 medium supplemented with 10% FBS and 50 μg/ml gentamicin. The cultures were observed daily by inverted phase-contrast microscopy. The culture medium was replaced 3 times a week. After primary cultures became confluent, the cells were detached by trypsin (0.05%)-EDTA (0.02%) solution, counted, centrifuged, re-suspended, diluted with a ratio 1:2-1:4, and seeded into culture flasks for subculture. Cells were passed routinely by a dilution of 1:2 to 1:4 at an interval for 4-7 days.

### Measurement of Proliferation of Cultured Articular Chondrocytes

Cell counting was used to evaluate the effect of the test substance on growth of articular chondrocytes. Chondrocytes were plated in 24-well plates at a density of 1×10⁴ cells per well. After 24 hours, medium was replaced by the testing media. The media was changed every three days. After six days, the cells were detached with trypsin-EDTA solution and neutralized with culture medium with 10% serum. The cell suspension was centrifuged and the pellet was resuspended in 1 ml F12 medium. 20 μl of the cell suspension was collected into the tip of a Pasteur pipette and transferred to a hemocytometer. The hemocytometer was observed under an optical microscope. Cells falling in four 1 mm³ areas bounded by three parallel lines were counted. The cell number was obtained using Formula I shown below (Freshney R I. Culture of Animal cells, 2nd edi. Willey-Liss, New York, 1987). The average of four counts was calculated. Triplicate samples were assayed in all experiments.
c=n×10⁴ FORMULA I
Where c=concentration (cells/ml), n=number of cells counted.

### RESULTS

### Effects of Various Basic Culture Media on the Proliferation of Cultured Articular Chondrocytes

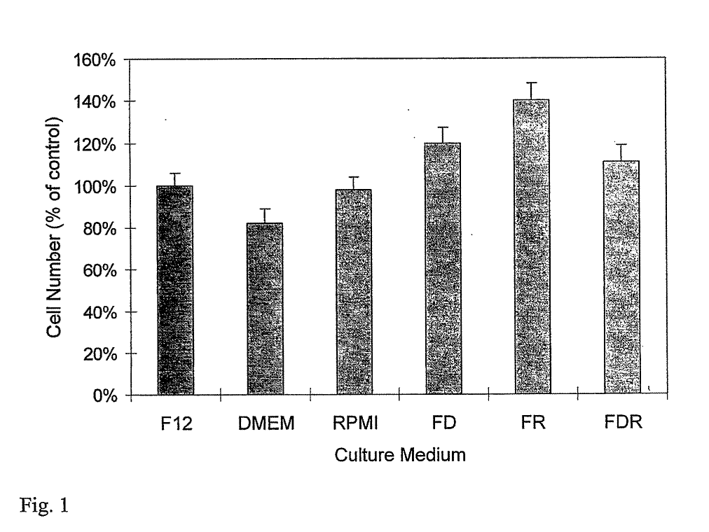

Chondrocytes grew better in the F-12 medium with RPMI medium (1:1)(FR) than in the F-12 medium alone (F12), DMEM medium alone (DMEM), RPMI medium alone (RPMI) and F-12, DMEM, RPMI mixed medium (1:1:1) (FDR) (all media were tested with 10% FBS) (FIG. 1). The difference of cell number between FR and FD were statistically significant (0.05>P>0.01). The difference of cell number between FR and F12, DMEM, RPMI or FDR were statistically very significant (P<0.01).

### Effects of bFGF on Cell Growth of Cultured Articular Chondrocytes

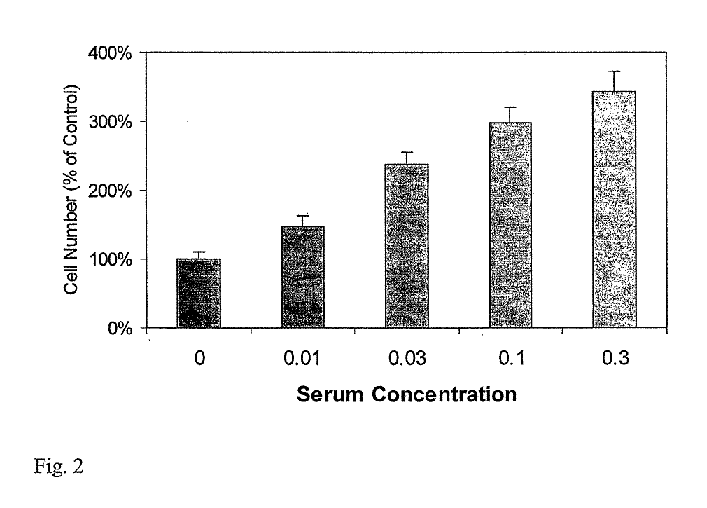

Cells cultured with serum-deleted medium grew very slowly. FBS showed dose-dependent growth stimulating effects (from 1% to 30%) on the cultured chondrocytes (FIG. 2). FBS at concentrations of 1%, 3%, 10% and 30% significantly stimulated the growth of cultured chondrocytes as compared with the controls (0.01<P<0.05 between 1% vs. the control; P<0.01 at all other groups). A statistically significant difference between the cell number of chondrocytes cultured with various concentrations of serum up to 10% FBS (P<0.01 between 1% vs. 3%, 0.01<P<0.05 between 3% vs. 10%). The cell number of chondrocytes cultured with 30% FBS was greater than that of cells cultured with 10% FBS. However, the difference was not statistically significant (P>0.05).

### Effects of bFGF on Cell Growth of Cultured Articular Chondrocytes

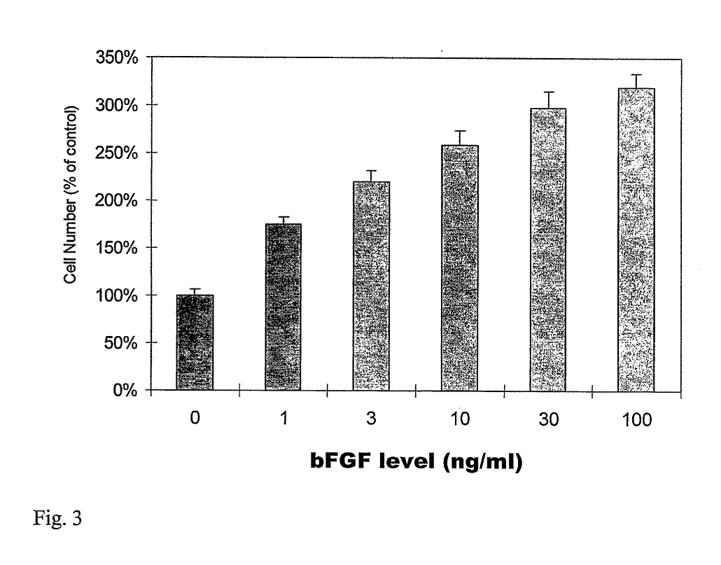

Addition of bFGF to the culture medium at concentrations of 1-100 μg/ml caused a dose dependent stimulation of cell growth (FIG. 3). Cell number of articular chondrocytes cultured with all tested concentration of bFGF was significantly greater than that of the controls (P<0.01 at all concentrations). Number of cells cultured with 10 μg/ml and 30 μg/ml bFGF was 259% and 298% of cells without bFGF.

### Effects of HGF on Cell Growth of Cultured Articular Chondrocytes

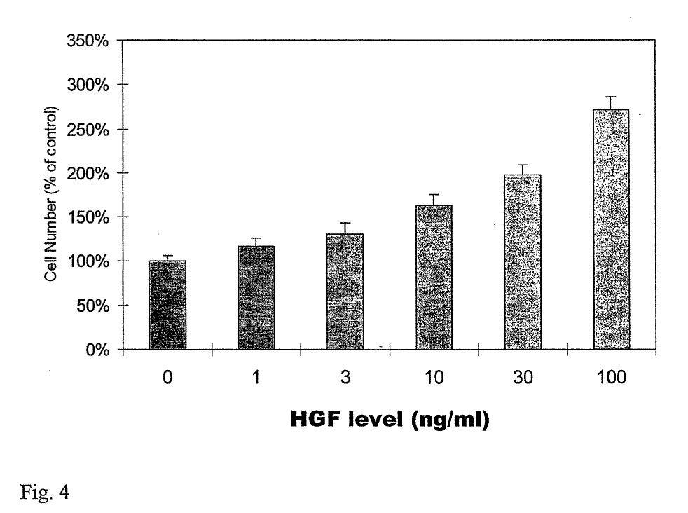

HGF at concentrations of 1-100 μg/ml caused a dose dependent stimulation of cell growth (FIG. 4). Cell number of articular chondrocytes cultured with 1 μg/ml was not significantly different from the controls (P>0.05). Cell number at all other tested concentrations of HGF was significantly greater than that of the controls (0.01<P<0.05 at a concentration of 3 μg/ml and P<0.01 at 10-100 μg/ml). Cell number of chondrocytes cultured with 100 μg/ml HGF was 271% of cells cultured without HGF.

### Effects of PDGF on Cell Growth of Cultured Articular Chondrocytes

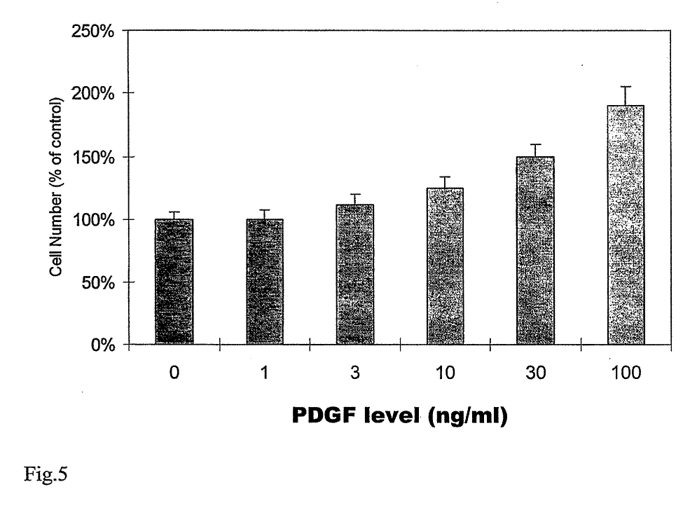

PDGF at concentrations of 1-100 μg/ml caused a dose dependent stimulation of cell growth (FIG. 5). Cell number of articular chondrocytes cultured with 1-3 μg/ml was not significantly different from the controls (P>0.05). Cell number at all other tested concentrations of PDGF was significantly greater than that of the controls (0.01<P<0.05 at a concentration of 10 μg/ml and P<0.01 at 30-100 μg/ml). Number of cells cultured with 30 μg/ml and 100 μg/ml PDGF was 150% and 190% of cells without PDGF, respectively.

### Effects of Insulin on Cell Growth of Cultured Articular Chondrocytes

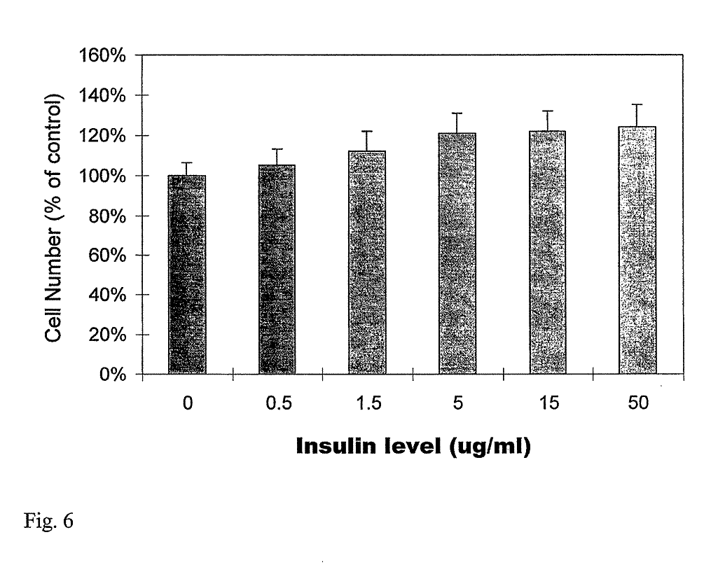

Insulin at concentrations of 5-50 μg/ml caused a significant stimulation of cell growth (FIG. 6). Cell number of chondrocytes cultured at 5-50 μg/ml insulin was significantly greater than that of the controls (0.01<P<0.05), whereas cell number of chondrocytes cultured with 0.5-1.5 μg/ml was not significantly different from the controls (P>0.05).

### Effects of Combination of Various Supplements on the Growth of Cultured Articular Chondrocytes

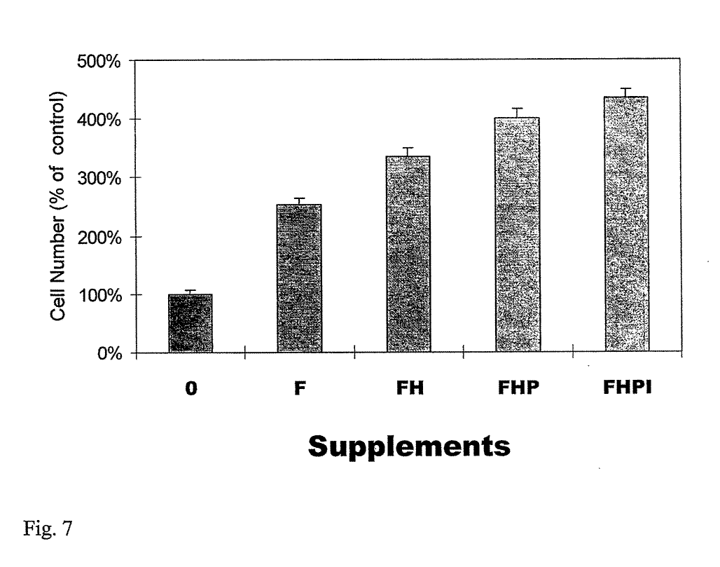

In articular chondrocytes cultured with F12-RPMI medium, adding of 15 μg/ml of bFGF caused a very significantly increase of cell number with 6 days (P<0.01), further addition of HGF (100 μg/ml) caused a 32% increase of cell growth (FH) (P<0.01 as compared with cells cultured with bFGF alone). In cells cultured with FH medium, adding of 50 μg/ml PDGF caused another significant increase of cell growth (12%, P<0.01) (FHP). Finally addition of 20 μg/ml of insulin to the FHP medium caused a small but significant increase of cell growth (P<0.05) (FIG. 7).

### Comparison of Hu60 Medium to Other Culture Media

The growth of articular chondrocytes cultured with Hu60 medium is compared to their growth in other kinds of medium.

The culture medium described in U.S. Pat. No. 6,150,163 (G medium) comprises DMEM, RPMI, F12 medium (1:1:1), with 1% ITS, penicillin (100 U/ml), streptomycin 100 ug/ml), hydrocortisone (40 μg/ml), bFGF 10 μg/ml), IGF-1 1 ng/ml and fibronectin 5 ug/ml.

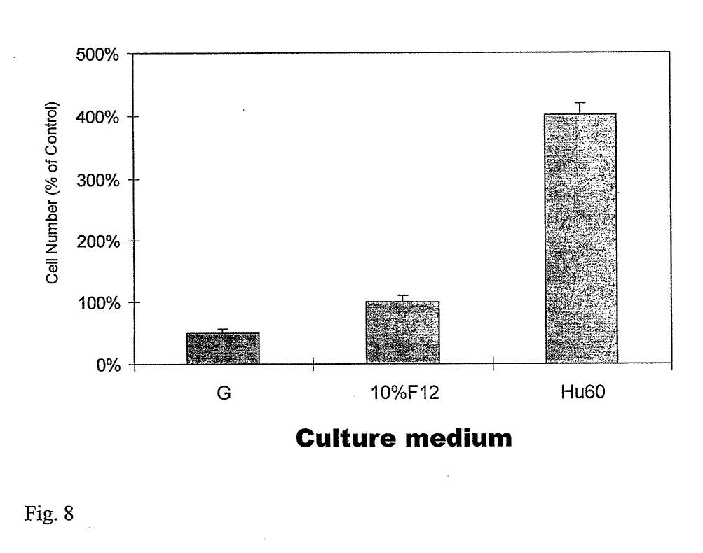

Articular chondrocytes cultured with Hu60 medium grew better than cells cultured in F12 medium with 10% FBS and G medium. Cell number of articular chondrocytes cultured with Hu60 medium was significantly greater than that cultured in F12 medium with 10% FBS and G medium (P<0.01). Cells grew very slowly in G medium and the cell number of cells cultured with G medium was very significantly less than that cultured with F12 medium with 10% FBS (P<0.01) (FIG. 8), indicating that chondrocytes grew very poor in culture medium without serum even in the presence of many growth stimulating factors.

### Comparison of Long-Term Effects on Cell Proliferation by Hu60 Medium with Other Culture Media

The long-term results of chondrocytes cultured with F12 medium with 10% serum, G medium and Hu60 medium were compared for three different cell lines. In each cell line, the chondrocytes were seeded into three flasks at the early subculture, and cultured with 3 different media. Cells were incubated and subcultured continuously, until senescence. Population doubling (PD) of each generation was calculated from the number of cells plated and the number of cells harvested using the following formula²²:
PD=(logNt−logNo)/log2 Formula II
No=cell number at the time of plating
Nt=cell number at the time of harvesting

The cumulative population doublings of a cell line cultured with a given culture medium is the sum of PD in all generations (Hu D N, Ritch R, McCormick S A, Pelton-Henrion K. Isolation and cultivation of human iris pigment epithelium. Invest Ophthalmol Vis Sci 1992;33:2443-53).

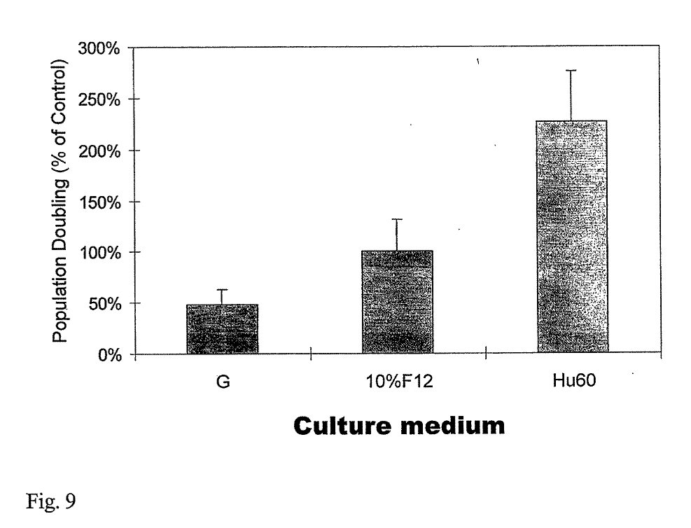

Long-term effect of these media on the growth of chondrocytes was compared. Chondrocytes cultured with Hu60 medium could divide 25.9 times (cumulative population doubling) before senescence, which was very significantly more than that cultured with F12 medium with 10% FBS (11.4) and G medium (5.43)(P<0.01). chondrocytes cultured in G medium grew slowly and the cell growth stopped within 2-3 generations and only divided 5.5 times, which was significantly less than that cultured with F-12 medium with 10% FBS (FIG. 9).

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A composition for culturing a population of chondrocytes comprising basal medium, serum, antibiotic, hormone, and growth factor, wherein the growth factor comprises PDGF and HGF.

2. The composition of claim 1, wherein the chondrocytes are selected from the group consisting of articular chondrocytes, auricular chondrocytes, nasal chondrocytes, and chondrocostal chondrocytes.

3. The composition of claim 1, wherein the serum is selected from the group consisting of human serum, bovine serum, newborn bovine serum, fetal bovine serum, porcine serum, equine serum, and a combination thereof.

4. The composition of claim 1, wherein the basal medium is selected from the group consisting of Ham's F12, RPMI, DMEM, and a combination thereof.

5. The composition of claim 4, wherein the basal medium is a combination of Ham's F12 and RPMI in a ratio of about 1:1.

6. The composition of claim 1, wherein the growth factor is selected from the group consisting of bFGF, HGF, PDGF, EGF, IGF, TGF-β, and a combination thereof.

7. The composition of claim 1, wherein the hormone is selected from the group consisting of androgen, mineralocorticoid, glucocortoid, insulin, hydrocortisone, estradiol, progestin, growth hormone, and a combination thereof.

8. A method of obtaining a proliferating population of chondrocytes, the method comprising isolating chondrocytes from a donor and culturing the chondrocytes using the composition of claim 1.

9. The method of claim 8, wherein the chondrocytes are isolated from a cartilage sample.

10. The method of claim 9, wherein the cartilage sample is treated enzymatically to dissociate chondrocytes from the cartilage sample.

11. A method of providing a subject in need of cartilage repair with a proliferating population of chondrocytes, the method comprising isolating chondrocytes from said subject, culturing the isolated chondrocytes using the composition of claim 1 to obtain a proliferating population of cultured chondrocytes; and applying the cultured chondrocytes to the cartilage requiring repair.

12. The method of claim 12, wherein the cultured chondrocytes are applied to surface of, or in the local environment of the damaged articular cartilage.

13. A composition comprising a population of chondrocytes cultured in a medium comprising basal medium, serum, antibiotic, hormone, and growth factor, wherein the growth factor comprises PDGF and HGF, wherein the cultured chondrocytes exhibit a number of divisions before senescence, wherein the number of divisions is at least 10.

14. The composition of claim 13, wherein the number of divisions is at least 20.

15. A composition for culturing a population of chondrocytes comprising Ham's F12, fetal bovine serum, glutamine, gentamicin, insulin, and growth factor, wherein the growth factor comprises bFGF, PDGF and HGF.
